(19) United States
(12) Patent Application Publication
(10) Pub. No.: US 2013/0305813 A1
(43) Pub. Date: Nov. 21, 2013

(54) SUBLIMABLE AROMATIC COMPOUND REMOVING UNIT FOR PROCESS GAS ANALYZING DEVICE, AND PROCESS GAS ANALYZING DEVICE INCLUDING THE SAME

(71) Applicant: HORIBA, Ltd., Kyoto-shi (JP)

(72) Inventors: Shigeyuki Akiyama, Kyoto-shi (JP); Yuki Imamura, Kyoto-shi (JP)

(73) Assignee: HORIBA, Ltd., Kyoto-shi (JP)

(21) Appl. No.: 13/898,268

(22) Filed: May 20, 2013

(30) Foreign Application Priority Data
May 18, 2012 (JP) 2012-114701

(51) Int. Cl. B01J 20/281; G01N 33/00

(52) U.S. Cl. CPC B01J 20/281; G01N 33/0014
USPC 73/31.07; 502/416

(57) ABSTRACT

A process gas analyzing device is provided. The process gas analyzing device may include a sample gas introducing unit configured to introduce a process gas including a sublimable aromatic compound; a sublimable aromatic compound removing unit configured to remove the sublimable aromatic compound; a process gas pretreatment unit arranged between the sample gas introducing unit and the sublimable aromatic compound removing unit; and a gas analyzing unit arranged in a downstream position of the sublimable aromatic compound removing unit. The sublimable aromatic compound removing unit may include an adsorbent of activated carbon including a non-volatile acid.

### BACKGROUND OF THE INVENTION

1. Field of the Invention

The invention relates to a sublimable aromatic compound removing unit for a process gas analyzing device, and a process gas analyzing device including the sublimable aromatic compound removing unit.

2. Description of the Related Art

In a continuous analysis of a gas generated from a high temperature coal gasification furnace, the gas includes a lot of dust and volatile organic compounds such as naphthalene and the compounds reacted with naphthalene, and thus the dust and the volatile organic compounds are removed from the gas by a water-washing type sampling device before analysis. The gas is also cooled down and dehumidified by the water-washing type sampling device for analysis. In the water-washing and sampling process of the gas, a sample gas water-washing method using a water-ejector (see FIG. 11) or a water-bubbler (see FIG. 12) has been used.

Japanese Laid-Open Patent publication No. 2008-163256 discloses that naphthalene can be removed with activated carbon. However, when the sample gas is pretreated with the activated carbon, the target gas is also adsorbed in the activated carbon. Therefore, the activated carbon cannot be used in the pretreatment step.

### SUMMARY OF THE INVENTION

The inventors of the present invention have recognized that a high concentration of naphthalene in the sample gas sometimes causes problems when requiring higher accuracy in the analysis of the gas, as the concentration of naphthalene cannot be decreased to less than the concentration under the saturated vapor pressure at the temperature of washing-water in the conventional measurement device, because naphthalene is a volatile organic compound and has a sublimable property. Additionally, in the conventional measurement device, the ratio in volume (g/w) of the quantity of sample gas flow (g) to the quantity of washing-water flow (w) at 25° C. and 1 atm is usually 1 or less, and thus the target gas in the sample gas may be dissolved in the washing-water and result in a dissolution loss and the oxygen dissolved in the washing-water may be degassed in the sample gas. In that case, the effects of the dissolution loss of the target gas and the degassing of dissolved oxygen on the measurement value sometimes cannot be ignored when requiring higher accuracy in the analysis of the gas.

Specifically, when the O₂ concentration in the gas from a coal gasification furnace is actually in the vicinity of 0.5%, the O₂ concentration in the sample gas is sometimes measured as 1.5% after water-washing of the sample gas by a washing tower. The measured O₂ concentration is three times of the actual O₂ concentration, and the measured value of O₂ concentration runs over the predetermined monitoring upper limit concentration. On the other hand, CO₂ concentration indicates the amount of heat generation, so that CO₂ is an important measuring target gas when the gas made of coal is used as fuel. However, CO₂ concentration of 10-20% in the sampling gas is sometimes reduced by half after the treatment with the washing tower. Accordingly, it is sometimes difficult to monitor the efficiency of the coal gasification reaction correctly and to continue safe operation in a major plant.

In view of the above-mentioned problems of the conventional art, an object of the present invention is to provide a process gas analyzing device, and a sublimable aromatic compound removing unit used therein, to reduce the dissolution loss of soluble gas, wasted washing-water, and error due to degassing of dissolved gas.

After active studies in order to achieve the object, the inventors discovered that the clogging of tubes and cooling devices due to the crystallization of sublimable aromatic compounds can be prevented by arranging a sublimable aromatic compound removing unit including a specific adsorbent at a later stage of the analyzing process, thereby conceiving of the invention.

In the present invention, a sublimable aromatic compound removing unit for a process gas analyzing device comprises an adsorbent of activated carbon including a non-volatile acid. The sublimable aromatic compound removing unit of the present invention can prevent the clogging of tubes and cooling devices due to the crystallization of sublimable aromatic compounds, and can reduce errors associated with sampling procedures by using the adsorbent of activated carbon including a non-volatile acid.

The non-volatile acid is preferably phosphoric acid.

In this case, by using phosphoric acid as the non-volatile acid, the handling can be effectively easier and the effect on the target materials can be reduced.

The adsorbent preferably includes 0.5% or more by weight of the non-volatile acid.

In this case, adsorption of the target gas such as CO₂ to the adsorbent can be more effectively prevented and the response delays can be more effectively decreased.

The adsorbent preferably includes 50% or less by weight of the non-volatile acid.

In this case, the removal rate of the sublimable aromatic compound can be further increased and the clogging of tubes and cooling devices due to the crystallization of the sublimable aromatic compounds can be more effectively prevented.

Preferably, the adsorbent further includes 10-50% by weight of water.

In this case, adsorption of the target gas such as CO₂ can be more effectively prevented due to the generation of acidic solution resulted from dissolution of the non-volatile acid into the water in the adsorbent.

Further, according to the present invention, a process gas analyzing device comprises:

a sample gas introducing unit configured to introduce a process gas including a sublimable aromatic compound;

a sublimable aromatic compound removing unit configured to remove the sublimable aromatic compound, the sublimable aromatic compound removing unit comprising an adsorbent of activated carbon including a non-volatile acid;

a process gas pretreatment unit arranged between the sample gas introducing unit and the sublimable aromatic compound removing unit; and

a gas analyzing unit arranged in a downstream position of the sublimable aromatic compound removing unit.

According to the configuration of the process gas analyzing device of the present invention, a continuously stable measurement of the process gas is made possible at any time of the year.

The process gas pretreatment unit is preferably a water-washing unit.

In this case, the concentration of the sublimable aromatic compounds in the sample gas can be more effectively reduced.

The water-washing unit preferably has a volume ratio (G/W), which is a ratio of the quantity of a sample gas flow (G) to the quantity of a washing-water flow (W), of 5 or more at 25° C. and 1 atm.

In this case, precise measurement of the concentration of the target gas can be taken more effectively by keeping the volume ratio (G/W) at a specific value.

Preferably, the process gas analyzing device further comprises a cooling unit between the process gas pretreatment unit and the sublimable aromatic compound removing unit.

In this case, the concentration of the sublimable aromatic compounds in the sample gas can be more effectively reduced.

The present invention contributes to improvement in precise measurement of the analyzing device for process gases, such as coal gas, and in safe plant operation, by selectively removing sublimable aromatic compounds such as naphthalene in gaseous form, thereby reducing errors and response delays in the analysis.

### BRIEF DESCRIPTION OF THE DRAWINGS

FIG. 1 is a diagram showing an example of the configuration of the process gas analyzing device of the present invention.

FIG. 2 is a diagram showing another example of the configuration of the process gas analyzing device of the present invention.

FIG. 3 is a diagram showing an example of the configuration of the sublimable aromatic compound removing unit of the present invention.

FIG. 4 is a diagram showing an example of a column configuration of the sublimable aromatic compound removing unit of the present invention.

FIG. 5 is a diagram showing an example use case of the gas flow in the process gas analyzing device.

FIG. 6 is a flow diagram showing an example of the steps of decreasing the concentration of naphthalene contained in the sampling gas in the process gas analyzing device.

FIG. 7 is a graph showing an example of the change of CO₂ recovering rate from the sampling gas against G/W in the water-washing unit.

FIG. 8 is a graph showing an example of the effect on the amount of oxygen which was dissolved in the washing-water and degassed into the sampling gas against G/W in the water-washing unit.

FIG. 9 is a graph showing an example of the relationship between the concentration of phosphoric acid and CO₂ response in the sublimable aromatic compound removing unit.

FIG. 10 is a graph showing an example of the relationship between the concentration of phosphoric acid and the naphthalene removing percentages in the sublimable aromatic compound removing unit.

FIG. 11 is a diagram showing a water jet type washer of the prior art.

FIG. 12 is a diagram showing a water-bubbler type washer of the prior art.

### DETAILED DESCRIPTION OF THE INVENTION

### Process Gas Analyzing Device

The process gas analyzing device of the present invention includes a sample gas introducing unit, a sublimable aromatic compound removing unit, a process gas pretreatment unit, and a gas analyzing unit. The sample gas introducing unit is configured to introduce a process gas including sublimable aromatic compounds, and the sublimable aromatic compound removing unit is configured to remove the sublimable aromatic compounds. The process gas pretreatment unit is arranged between the sample gas introducing unit and the sublimable aromatic compound removing unit. The gas analyzing unit is arranged in a downstream position of the sublimable aromatic compound removing unit. The sublimable aromatic compound removing unit has an adsorbent containing non-volatile acids.

Examples of the process gas to be analyzed by the process gas analyzing device in the present invention include a coke-oven gas, a coal gasification gas, a steel-manufacturing furnace gas, a petroleum refinery process gas, an organic waste gasification gas, and the like. These process gases contain sublimable aromatic compounds such as naphthalene, anthracene, and paradichlorobenzene. The sublimable aromatic compounds cause problems due to their sublimate character and aromatic character. The problem caused by the sublimate character is that the sublimable aromatic compounds flow in the gaseous phase and crystallize upon cooling the gas down. The problem caused by the aromatic character is deterioration of parts such as joints made of, specifically, plastics, such as polyvinyl chloride.

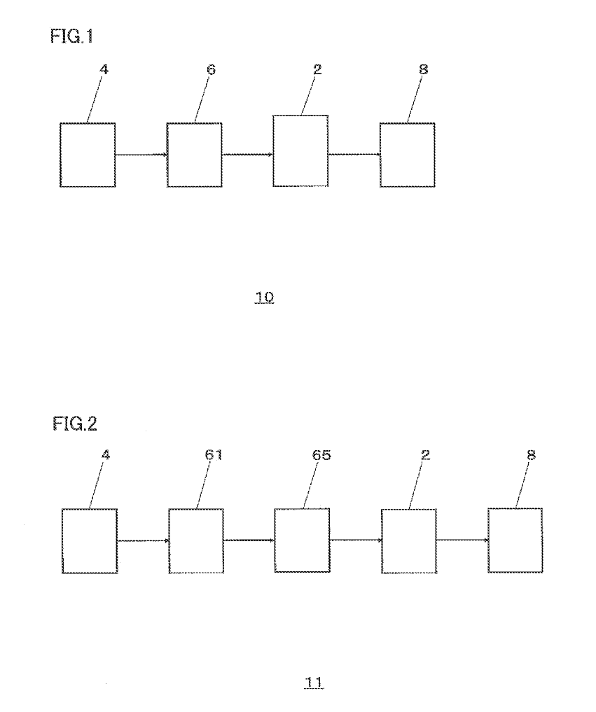

As shown in FIG. 1, a process gas analyzing device 10 of one example of the present invention includes a sample gas introducing unit 4, a process gas pretreatment unit 6, a sublimable aromatic compound removing unit 2, and a gas analyzing unit 8. Hereinafter, “the sublimable aromatic compound removing unit” is also called simply, “the removing unit.” FIG. 2 shows a configuration of another process gas analyzing device 11 of another example of the present invention. In the process gas analyzing device 11, the process gas pretreatment unit 6 is configured as a water-washing unit 61. In that case, a cooling unit 65 may be further arranged between the water-washing unit 61 and the removing unit 2. Examples of the cooling unit 65 may include a coiled condenser and a wet filter equipped with a coiled condenser. Using the cooling unit, the sublimable aromatic compounds may be removed more surely. The coil of the cooling unit may be made from 1 to 2 meters of stainless steel tube. The process gas can be cooled down to room temperature by using such a coil.

(1) Sample Gas Introducing Unit

The sample gas introducing unit introduces a process gas to the process gas analyzing device. Specifically, a probe tube may be used as the sample gas introducing unit. The sample gas introducing unit may also be provided with a heater which heats the sample gas introducing unit and keeps it warm. The sample gas introducing unit is inserted in a duct that carries the process gas, takes the process gas by aspiration, and introduces the process gas into the water-washing unit. The sample gas introducing unit is preferred to be made of inorganic material, for example metal such as stainless steel or ceramics, which has heat resistance and corrosion resistance. More preferably, the sample gas introducing unit may be made of a straight tube having 6 mm in outer diameter/4 mm in inner diameter, or 8 mm in outer diameter/6 mm in inner diameter, and 1-5 m in length. It is also preferred that the inner surface of the sample gas introducing unit has lubricity.

The sample gas introducing unit is preferred to be kept and used at 100-500° C. to avoid condensation of water vapor or compounds having lower boiling points, and to avoid naphthalene depositing and adhering to the sample gas introducing unit. When the inside of the duct has a high temperature of about 300-400° C., the sample gas introducing unit can have a high temperatures of 200° C. or more and can be kept at this temperature by taking the process gas from the duct at its unchanged temperature. Usually, the heater is preferred to be complementarily provided to heat the sample gas introducing unit upon changing the temperature of the process gas, starting combustion facilities, changing load, or operating in winter or in cold regions. The heater is preferred to cover all of the outer periphery of the sample gas introducing unit so as to heat it efficiently and to reliably keep the heat.

(2) Process Gas Pretreatment Unit

The process gas pretreatment unit removes the sublimable aromatic compounds from the sample gas introduced from the sample gas introducing unit. Removing methods are not particularly limited, as long as the concentration of the sublimable aromatic compounds contained in the sample gas can be decreased to be less than or equal to the concentration under the saturated vapor pressure at the processing temperature of the sublimable aromatic compounds removing unit, which is arranged in a downstream position of the process gas pretreatment unit. The process gas pretreatment unit may be any unit just having a cooling unit that can cool down the sample gas, make the sublimable aromatic compounds deposit, and can remove the deposited sublimable aromatic compounds. The temperature of the sample gas after treatment with the process gas pretreatment unit may be 40° C. or less, and preferably may be the temperature of the washing-water (15-25° C., for example). The process gas pretreatment unit is preferred to be a water-washing unit.

(2-1) Water-Washing Unit

The water-washing unit, such as a washing tower, pours or supplies water to the sample gas to clean and cool down the gas. The water may be supplied as mist. The water for cleaning the gas may be supplied from a tap of running water which is supplied from the public or industrial water supply. The flow rate (G) of the sample gas may be 2-5 L/min at dry, 25° C. and 1 atm. The ratio (G/W) of the quantity of a sample gas flow (G), at dry, 25° C. and 1 atm, to the quantity of a washing-water flow (W) at 25° C. and 1 atm is preferably 5 or more, specifically 5-15, and more preferably 10 or more. The ratio (G/W) of less than 5 is not preferred because concentration measurements of water-soluble gas such as CO₂ contained in the sample gas may be sometimes imprecise due to high dissolution of the soluble gas into the washing-water. Also, in such a case, it is not preferred because the concentration measurements of oxygen may be sometimes imprecise due to the contamination of oxygen, which has been dissolved in the washing-water and is degassed into the sample gas. When the ratio (G/W) is more than 15, the sample gas may not be cleaned and cooled down enough, so that the removal of the sublimable aromatic compounds may be sometimes insufficient. The mixture of the sample gas and the washing-water after the treatment by the water-washing unit is separated by, for example, a gas-liquid separator, and then, the sublimable aromatic compounds deposited are recovered to a drain pot with the washing-water.

(3) Sublimable Aromatic Compound Removing Unit

The sublimable aromatic compound removing unit of the present invention includes an adsorbent having non-volatile acids. The sublimable aromatic compound removing unit is preferred to remove the sublimable aromatic compounds in the gas phase. The removal of the gaseous sublimable aromatic compounds makes it easier to avoid contamination of oxygen to the process gas and to avoid dissolution loss of the soluble gas such as CO₂. One preferable example of the sublimable aromatic compound removing unit is a column packed with an adsorbent that removes the sublimable aromatic compounds. The volume of the column and the amount of adsorbent to be packed in the column may differ depending on the concentration and flow rate of the sublimable aromatic compounds to be treated and the hours of use of the column. For example, the column volume may be 200-400 mL, and the amount of packed adsorbent may be 50-200 g. The column may be a one-series column, a two-series column, a two-series of a pair of columns, or the like. The two-series column can be used advantageously in a continuous measurement.

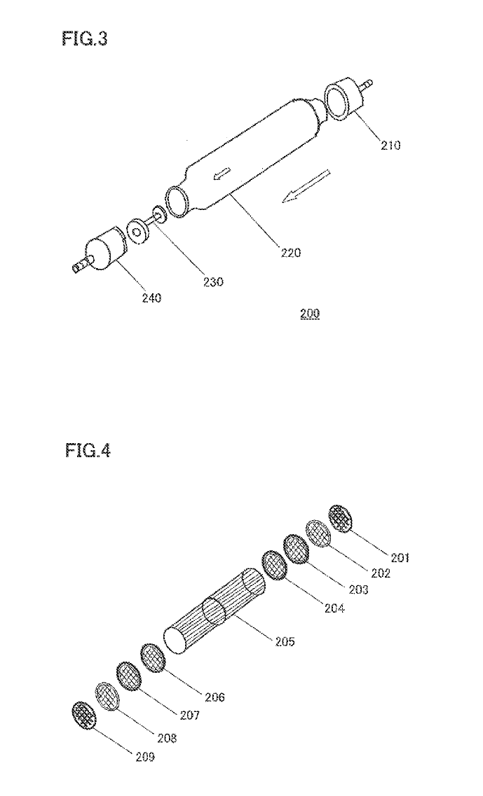

FIG. 3 shows an example of the configuration of the sublimable aromatic compound removing unit 200 of the present invention. The sublimable aromatic compound removing unit 200 includes a cap 210 at a unit inlet (an inlet cap), a column body 220, a spacer 230, and a cap 240 at a unit outlet. An opening is formed in each of the caps 210 and 240 to allow gas to pass through. As shown in FIG. 3, the caps 210 and 240 each have a part having a smaller diameter, and a through hole is formed in each of these parts. Arrows in FIG. 3 indicate directions of the gas flow. The spacer 230 serves as a drain trap, and has a space for storing water. The water content of the adsorbent may be kept during storage by attaching the caps at the unit inlet and outlet. Gas inlet and outlet of the parts having the smaller diameter may be respectively sealed by a lid during storage. The cap 240 may be adhered with the spacer 230 in advance, for example with a lower portion of the spacer 230.

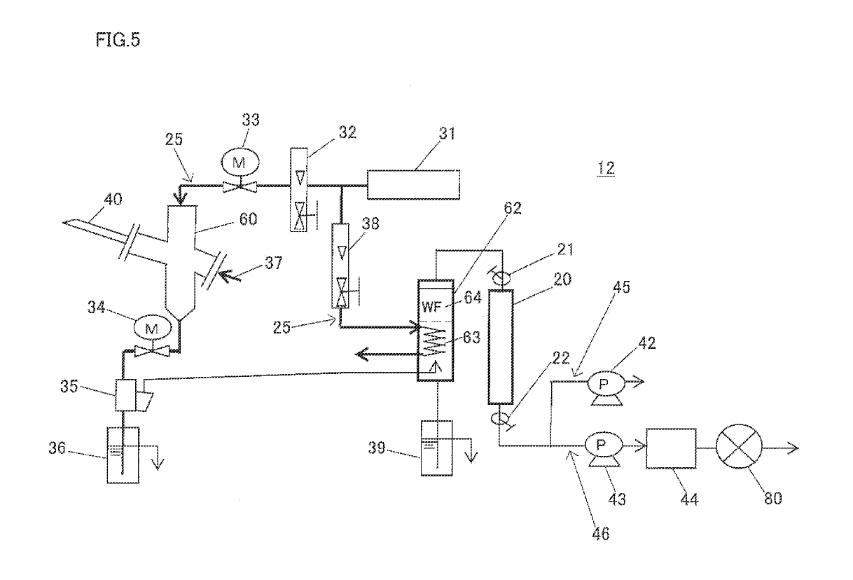

As shown in FIG. 4, a plastic net 201, filter layers 202-204, an adsorbent particles layer 205, filter layers 206-208, and a plastic net 209 are configured in this order, from the side of the inlet cap in the column 220. The plastic nets may prevent clogging due to the precipitate of the sublimable aromatic compounds such as naphthalene, and the like. The opening sizes of the filter layers 203, 204, 206 and 207 may be smaller than those of the filter layers 202 and 208.

Adsorbent

In the present invention, the sublimable aromatic compound removing unit includes activated carbon having non-volatile acids as the adsorbent. Examples of the non-volatile acids include phosphoric acid, sulfuric acid and permanganic acid. Further, the examples of the non-volatile acids may include salts of these acids. Phosphoric acid is preferable as the non-volatile acid in view of handling or effect on the measuring objects. The sublimable aromatic compound removing unit may include another adsorbent in combination with the above-mentioned adsorbent unless it has an adverse effect on the present invention.

The concentration of the non-volatile acids in the adsorbent is preferably 0.5% by weight or more, specifically 0.5-50% by weight, more preferably 5-50% by weight, and further preferably 10-50% by weight. Containing no non-volatile acid in the adsorbent causes poor CO₂ responsiveness in 90%. Containing less than 0.5% by weight of the non-volatile acids in the adsorbent sometimes may cause insufficient CO₂ responsiveness in 90%. Containing more than 50% by weight of the non-volatile acids in the adsorbent sometimes may cause insufficient removal of the sublimable aromatic compounds.

The activated carbon containing the non-volatile acids can be prepared by conventional manufacturing processes, for example by immersing activated carbon into a solution of the non-volatile acids and a solvent, removing the carbon from the solution and then removing the residual solvent from the resulting carbon.

In case of using the two-series of a pair of columns, the concentrations of the non-volatile acids in the adsorbents packed in the two columns may be the same or may differ from one another. For example, the two columns respectively packed with adsorbents having non-volatile acid concentrations of 0% and 5%, 5% and 10%, 10% and 15%, 15% and 20%, or 20% and 30%, by weight can be used.

The adsorbent is preferred to further contain water. In this case, adsorption of the measuring gas such as CO₂ can be effectively prevented due to generation of an acidic solution by dissolution of the non-volatile acids into the water. The amount of the water is preferably 0.5-50% by weight, more preferably 20-50% by weight. Containing less than 0.5% by weight of the water sometimes may cause insufficient CO₂ responsiveness in 90%. Containing more than 50% by weight of the water sometimes may cause insufficient flow of the gas. Further, when the concentration of the non-volatile acids in the adsorbent is less than 10% by weight, the amount of the water in the adsorbent is preferably 40-50% by weight.

A method for adding the water to the adsorbent is not limited in particular, as long as the water can be added to the adsorbent. For example, the water in a predetermined volume may be sprayed to the adsorbent. In another example, the adsorbent may be immersed in the water, the immersed adsorbent may be removed from the water, and then the excess water may be removed from the resulting adsorbent. The water may be removed, for example at 75-100° C. in 2-5 hours.

(4) Cooling Unit

In case of the process gas pretreatment unit being the water-washing unit, a cooling unit may be arranged between the process gas pretreatment unit and the sublimable aromatic compound removing unit. With the cooling unit, the process gas may be more reliably cooled down to the washing-water's temperature, preferably 25° C., so that the concentration of the sublimable aromatic compounds can be decreased to less than or equal to the concentration under the saturated vapor pressure at the washing-water temperature, even if the cleaning or cooling of the process gas is insufficient in the water-washing unit.

Preferably, a coiled condenser made of stainless steel may be used as the cooling unit. In the coiled condenser, the water for cooling the gas may be supplied from a tap of running water which is supplied from the public or industrial water supply to the coil, and the gas passing along the outside of the coil is cooled down. The temperature of the cooling water is preferably normal temperature, for example 5-30° C., preferably 25° C. or less. The gas temperature at the outlet of the cooling unit is preferably normal temperature, for example 5-30° C., preferably 25° C. or less. The sublimable aromatic compounds deposited and adhered to the outside of the coil may be removed by generating a vibration of the coil to release the adhered materials and by washing the material with the water or the like. According to this procedure, the deposited sublimable aromatic compounds may be recovered in the drain pot. A wet filter may be further arranged in a downstream position of the coil condenser to catch the deposited sublimable aromatic compounds and the dust, more reliably. Using the cooling unit may allow the concentration of the process gas to decrease more reliably to less than or equal to the concentration under the saturated vapor pressure at the wash-water temperature, for example 5-30° C., preferably 25° C. or less.

(5) Gas Analyzing Unit

The gas analyzing unit analyzes concentrations of O₂, CO, CO₂, CH₄, or the like. The measurement of O₂ concentration is important for safe process operation. A situation with a high O₂ concentration is dangerous, so that an O₂ concentration monitoring may be set to have an upper limit of O₂ concentration at about 0.5 vol %. On the other hand, the measurement of CO₂ concentration is important for indicating an index for the amount of heat generation. These measurements are used to control processes, so a rapid response, for example a response within 2 minutes or less, is needed for these measurements.

The gas is analyzed at a measuring temperature below room temperature, for example 0-15° C., preferably around 5° C. The sample gas to be analyzed is cooled down to the measuring temperature in the gas analyzing unit. In this cooling procedure, when the sample gas contains the sublimable aromatic compounds having a concentration higher than that under the saturated vapor pressure at the measuring temperature, the sublimable aromatic compounds are deposited during the cooling procedure, and may clog up the gas analyzing unit and make it difficult for the gas analyzing unit to continue the measurements. Therefore, the concentration of the sublimable aromatic compounds is preferred to be the concentration under the saturated vapor pressure at the measuring temperature or less.

### EXAMPLES

Hereinafter, specific use case examples of the process gas analyzing device will be described with reference to drawings, but the present invention is not limited to these use case examples.

FIG. 5 shows a process gas analyzing unit 12 as used in one example. The process gas analyzing unit 12 included a probe tube 40 as a sample gas introducing unit, water-washing unit 60 as a process gas pretreatment unit, a wet filter equipped with a coiled condenser 62, a gaseous naphthalene removing unit 20 as a sublimable aromatic compound removing unit, and an analyzer 80. In the process gas analyzing unit 12, the naphthalene concentration of the sample gas was decreased to a predetermined concentration by removing naphthalene through the water-washing unit 60, the wet filter equipped with the coiled condenser 62, and the gaseous naphthalene removing unit 20.

Sample Gas Introducing Unit

The probe tube 40, which served as a sample gas introducing unit, was inserted in a duct carrying a process gas, and drew in the process gas by aspiration and introduced the process gas to the water-washing unit 60. The probe tube 40 was made from a stainless steel straight tube having 6 mm in outer diameter/4 mm in inner diameter, and about 1-5 m in length. The probe tube 40 was kept and used at 100-500° C. The temperature inside the duct was about 300-400° C., and the temperature of the probe tube 40 was kept at 200° C. or more by taking the process gas at its unchanged temperature.

Process Gas Pretreatment Unit

The heated process gas drawn in by aspiration was introduced to the water-washing unit 60, which served as the process gas pretreatment unit, arranged in a downstream position of the sample gas introducing unit. The amount of the introduced process gas was 3 L/min, as calculated at 1 atm and 25° C. Various amounts of water for cleaning the gas were supplied to the water-washing unit 60 from a water supply line 31 of running water which is supplied to the public or industrial water supply, by opening a valve equipped with a flow meter 32 and a magnetically actuated valve 33. Dust and naphthalene deposited due to the cooling of the process gas were collected in the water.

The mixed fluid of the process gas and the water was introduced into a drain separator 35 through a magnetically actuated valve 34. The mixed fluid of the process gas and the water was separated into liquid and gas. The liquid was collected in the drain pot 36, and the gas was introduced to the wet filter equipped with the coiled condenser 62. The temperature of the process gas was about 40° C. near the motor valve 34. The water-washing unit 60 could be cleaned through purging with clean air from downstream 37 periodically. The dust and naphthalene adhering to the inside of the water-washing unit 60 could be removed by the air purging.

In the wet filter equipped with the coiled condenser 62, cooling water 25 was supplied to a coil 63 from a water supply line 31 by opening a valve equipped with a flow meter 38. Naphthalene was deposited from the process gas by cooling the process gas down to the temperature of the cooling water, specifically 25° C., and some of the deposited naphthalene was adhered to the outside of the coil. The deposited naphthalene was trapped in a drain pot 39 or a filter 64. The process gas that passed through the wet filter equipped with the coiled condenser 62 was introduced to the gaseous naphthalene removing unit 20, which served as a sublimable aromatic compound removing unit.

Sublimable Aromatic Compound Removing Unit

A naphthalene removing column (not shown) having the volume of about 300 mL was included in the gaseous naphthalene removing unit 20, which served as a sublimable aromatic compound removing unit. A plastic net, filter layers, an adsorbent, particles layer having 100 g of adsorbent, filter layers, a plastic net, and a drain trap structure were configured in the naphthalene removing column. Caps 21, 22 were provided at the column inlet and outlet, respectively. Adsorbent 1 of activated carbon containing a predetermined amount of phosphoric acid, and Adsorbents 2, 3 of activated carbons each containing a predetermined amount of phosphoric acid and further containing water were used. Activated carbons similar to Adsorbents 1-3 except for not containing phosphoric acid, i.e., the adsorbents containing zero amount of phosphoric acid, were prepared and used in Comparative Example.

Adsorbent 1 was prepared by immersing activated carbon in phosphoric acid solution, removing the carbon from the solution, and drying it in vacuum for 4 hours at 110° C. Adsorbent 2 was prepared by spraying the water to Adsorbent 1 at the ratio of 40 g water per 100 g of Adsorbent 1. Adsorbent 3 was prepared by immersing Adsorbent 1 in water for a few seconds, removing the Adsorbent 1 from the water, and treating it for 3 hours at 80° C. The amount of water in Adsorbent 3 was 53.3% by weight on average.

Gas Analyzing Device

0.5 L/min of the process gases, after passing through the naphthalene removing units respectively packed with the above-mentioned Adsorbents, were introduced respectively to an analyzer line 46 through a suction pump 43. 2.5 L/min of the residual process gas was carried to a bypass line 45 through a suction pump 42. The gas in the analyzer line 46 was cooled down to around 5° C. by an electronic cooler 44. The cooled gas was introduced to the analyzer 80 to measure concentrations of O₂ and CO₂. The electronic cooler 44 may include two coolers, and the cooler may be exchanged upon clogging of one cooler.

Naphthalene Concentration Decreasing Steps of Sample Gas

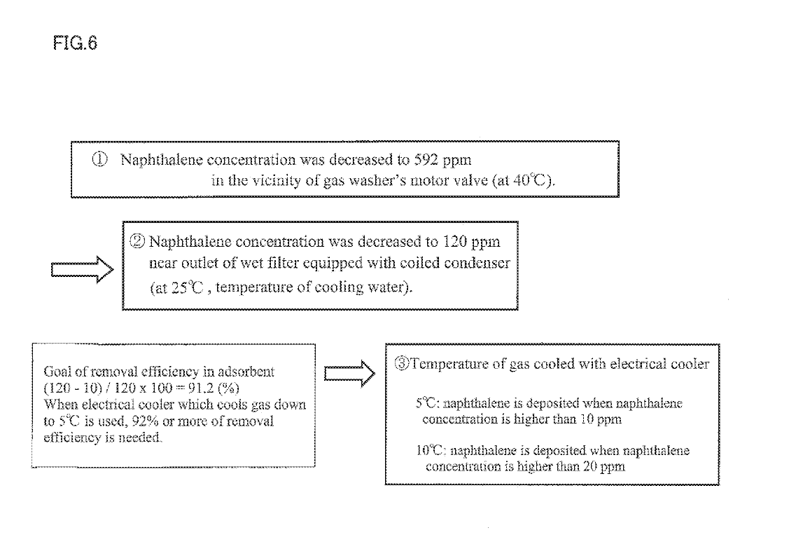

FIG. 6 shows steps for decreasing naphthalene concentration of sample gas. In step 1, the concentration of naphthalene was decreased to 592 ppm under the saturated vapor pressure at 40° C. which was the temperature of the sample gas around the outlet of the water-washing unit 60 (gas washer). Next, in step 2, the concentration of naphthalene was decreased to 120 ppm under the saturated vapor pressure at 25° C. which was the temperature of the sample gas around the outlet of the wet filter equipped with the coiled condenser 62.

After step 2, the cooled sample gas was further cooled down to around 5° C. or around 10° C. before and during measurement in the analyzer 80, as described below. In that case, the concentration of gaseous naphthalene contained in the sample gas needs to be decreased to 10 ppm or 20 ppm as a concentration under the saturated vapor pressure at around 5° C. or around 10° C. Naphthalene concentration of the sample gas being higher than that under the saturated vapor pressure at the measuring temperature causes problems as naphthalene is deposited and adhered to analyzer tubes. In step 3, to avoid these problems when using a cooler that cools the process gas down to around 5° C., 110 ppm (91.2%) of naphthalene had to be removed in the gaseous naphthalene removing unit 20 from the sample gas that passed through the wet filter equipped with the coiled condenser 62 and that contained 120 ppm of naphthalene. Therefore, in step 3, the goal of removal rate of naphthalene in the gaseous naphthalene removing unit 20 was 92% or more.

CO₂ Dissolution Loss at Water-Washing Unit

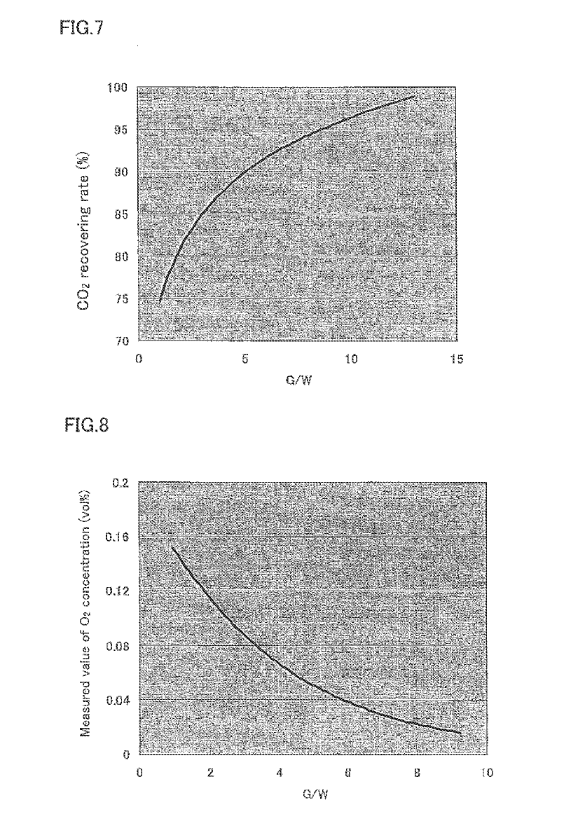

FIG. 7 shows the relationship between flow rate of the washing-water W and CO₂ recovering rate in the water-washing unit 60. The flow rate of the process gas G was 3 L/min, as calculated at 1 atm and 25° C. When the volume ratio (G/W) of the quantity of a sample gas flow (G) to the quantity of a washing-water flow (W) at 25° C. and 1 atm was 5 or more, the CO₂ dissolution loss was 10% or less. In other words, the CO₂ recovering rate was 90% or more. However, in additional view of preventing O₂ generation from the washing-water, it is preferred that the volume ratio (G/W) is 10 or more.

Effect of Degassing of Dissolved Oxygen at Water-Washing Unit

FIG. 8 shows an effect on degassing of dissolved oxygen at the water-washing unit 60. Here, nitrogen gas containing no oxygen was used instead of the process gas. The flow rate of nitrogen G was 3 L/min, as calculated at 1 atm and 25° C. When the volume ratio (G/W) of the quantity of the gas flow (G) to the quantity of a washing-water flow (W) at 25° C. and 1 atm was 5 or more, the amount of oxygen gas degassed from the washing-water and contained in the nitrogen gas was less than 0.05 vol %, and the generation of oxygen was suppressed.

Effect of Adding Phosphoric Acid and Water on CO₂ Response

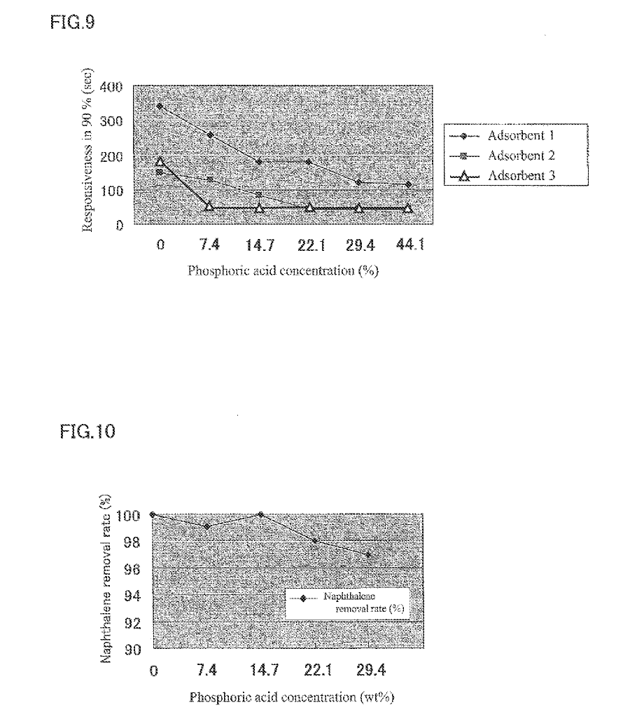

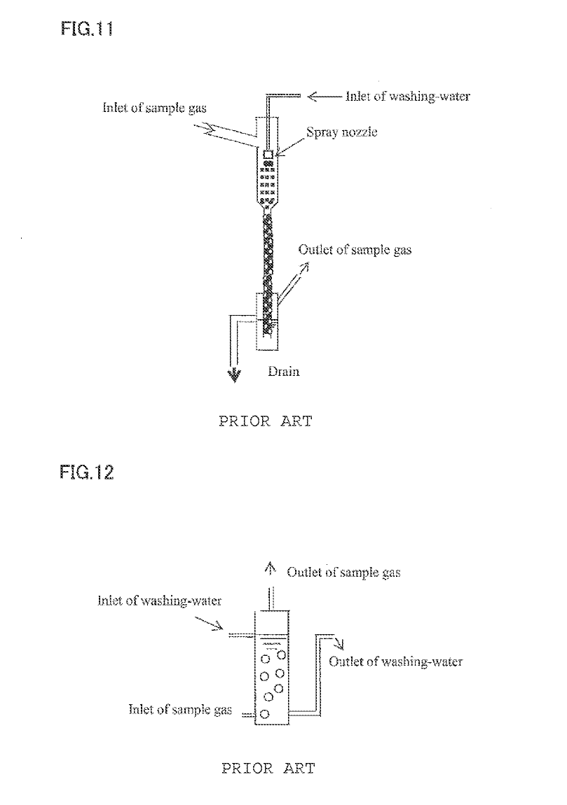

FIG. 9 shows a relationship between the contents of phosphoric acid and water in the adsorbent and CO₂ responsiveness in 90%. The content of phosphoric acid is indicated by weight %. In the sublimable aromatic compound removing unit, the responsiveness in 90% was 5-6 min when using only activated carbon in the column. The responsiveness in 90% was improved by the addition of phosphoric acid to activated carbon. By inclusion of water in the adsorbent, the responsiveness in 90% was further improved. The responsiveness in 90% was improved to 40-50 seconds even with a low concentration of phosphoric acid.

Effect of Adding Phosphoric Acid on Naphthalene Removal

A gas containing 100 ppm of naphthalene was carried at SV (Space Volume: the volume of gas flow passing through the adsorbent/the volume of the adsorbent)=4200 (hr⁻¹) to observe the effect of adding phosphoric acid on naphthalene removal. The results are indicated in FIG. 10. 96% or more of removal rate of naphthalene from the gas was secured with about 30% or less by weight of phosphoric acid concentration in the adsorbent. Further, paradichlorobenzene was removed in addition to naphthalene.

In the present invention, the gas at the outlet of the process gas pretreatment unit, such as the water-washing unit, is carried to the sublimable aromatic compound removing unit, after optionally passing though the cooler with the coil. Therefore, significant effects of the removal of the sublimable aromatic compounds in gas phase to a concentration lower than that under the saturated vapor pressure at the outside temperature are obtained, thereby preventing the clogging of the tubes and cooling devices due to the crystallization of the sublimable aromatic compounds.

In the above Example, the water-washing unit preferably had the volume ratio (G/W) of 5 or more, of the quantity of the sample gas flow (G) to the quantity of the washing-water flow (W), at 25° C. and 1 atm. However, a water-washing unit may be further provided with a system for temporarily increasing the water supply quantity so as to compulsorily remove dust or naphthalene adhering to walls of device tube when there is a risk of clogging occurring due to the dust or the like.

### BRIEF DESCRIPTION OF REFERENCE CHARACTERS

10, 11, 12 Process gas analyzing device
2, 200 Sublimable aromatic compound removing unit
20 Gaseous naphthalene removing unit
4 Sample gas introducing unit
40 Probe tube
6 Process gas pretreatment unit
60, 61 Water washing unit
62 Wet filter equipped with the coiled condenser
65 Cooling unit
8, 80 Gas analyzing unit

What is claimed is:

1. A sublimable aromatic compound removing unit for a process gas analyzing device, comprising an adsorbent of activated carbon including a non-volatile acid.

2. The sublimable aromatic compound removing unit of claim 1, said non-volatile acid being phosphoric acid.

3. The sublimable aromatic compound removing unit of claim 1, said adsorbent including 0.5% or more by weight of said non-volatile acid.

4. The sublimable aromatic compound removing unit of claim 1, said adsorbent including 50% or less by weight of said non-volatile acid.

5. The sublimable aromatic compound removing unit of claim 1, said adsorbent further including 10-50% by weight of water.

6. A process gas analyzing device comprising:
a sample gas introducing unit configured to introduce a process gas including a sublimable aromatic compound;
a sublimable aromatic compound removing unit according to claim 1 configured to remove said sublimable aromatic compound;
a process gas pretreatment unit arranged between said sample gas introducing unit and said sublimable aromatic compound removing unit; and
a gas analyzing unit arranged in a downstream position of said sublimable aromatic compound removing unit.

7. The process gas analyzing device of claim 6, said process gas pretreatment unit being a water-washing unit.

8. The process gas analyzing device of claim 7, said water-washing unit having a volume ratio (G/W), which is a ratio of a quantity of a sample gas flow (G) to a quantity of a washing-water flow (W), of 5 or more at 25° C. and 1 atm.

9. The process gas analyzing device of claim 6, further comprising a cooling unit between said process gas pretreatment unit and said sublimable aromatic compound removing unit.
